SEQUENCE LISTING (1 sequence)

SEQ ID NO: 1 - DNA, 2217 bp, synthetic construct
  source 1..2217: mol_type = other DNA; note = Plasmid GPC
ggatccatgggacaaatagtgacattcttccaggaagtgcctcatgtaatagaagaggtgatgaacattgttctcattgcactgtctgtactagcagtgctgaaaggtctgtacaattttgcaacgtgtggccttgttggtttggtcactttcctcctgttgtgtggtaggtcttgcacaaccagtctttataaaggggtttatgagcttcagactctggaactaaacatggagacactcaatatgaccatgcctctctcctgcacaaagaacaacagtcatcattatataatggtgggcaatgagacaggactagaactgaccttgaccaacacgagcattattaatcacaaattttgcaatctgtctgatgcccacaaaaagaacctctatgaccacgctcttatgagcataatctcaactttccacttgtccatccccaacttcaatcagtatgaggcaatgagctgcgattttaatgggggaaagattagtgtgcagtacaacctgagtcacagctatgctggggatgcagccaaccattgtggtactgttgcaaatggtgtgttacagacttttatgaggatggcttggggtgggagctacattgctcttgactcaggccgtggcaactgggactgtattatgactagttatcaatatctgataatccaaaatacaacctgggaagatcactgccaattctcgagaccatctcccatcggttatctcgggctcctctcacaaaggactagagatatttatattagtagaagattgctaggcacattcacatggacactgtcagattctgaaggtaaagacacaccagggggatattgtctgaccaggtggatgctaattgaggctgaactaaaatgcttcgggaacacagctgtggcaaaatgtaatgagaagcatgatgaggaattttgtgacatgctgaggctgtttgacttcaacaaacaagccattcaaaggttgaaagctgaagcacaaatgagcattcagttgatcaacaaagcagtaaatgctttgataaatgaccaacttataatgaagaaccatctacgggacatcatgggaattccatactgtaattacagcaagtattggtacctcaaccacacaactactgggagaacatcactgcccaaatgttggcttgtatcaaatggttcatacttgaacgagacccacttttctgatgatattgaacaacaagctgacaatatgatcactgagatgttacagaaggagtatatggagaggcaggggaagacaccattgggtctagttgacctctttgtgttcagtacaagtttctatcttattagcatcttccttcacctagtcaaaataccaactcataggcatattgtaggcaagtcgtgtcccaaacctcacagattgaatcatatgggcatttgttcctgtggactctacaaacagcctggtgtgcctgtgaaatggaagagaggcggtggcggtatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagatatc

(19) United States
(12) Patent Application Publication
(10) Pub. No.: US 2025/0114579 A1
(43) Pub. Date: Apr. 10, 2025

(54) DRUG DELIVERY SYSTEM, AND PREPARATION METHOD AND USE METHOD THEREOF

(71) Applicant: BEIHANG UNIVERSITY, Beijing (CN)

(72) Inventors: Ying WANG, Beijing (CN); Yubo FAN, Beijing (CN); Jiebo LI, Beijing (CN); Zhongyi SU, Beijing (CN); Yiting WANG, Beijing (CN); Daxin WU, Beijing (CN)

(73) Assignee: BEIHANG UNIVERSITY, Beijing (CN)

(21) Appl. No.: 18/377,883

(22) Filed: Oct. 9, 2023

(51) Int. Cl. A61M 37/00; A61K 31/7088; A61N 5/067; B82Y 5/00

(52) U.S. Cl. CPC A61M 37/0015; A61K 31/7088; A61N 5/067; B82Y 5/00; A61M 2037/0023; A61M 2037/0053; A61M 2037/0061

(57) ABSTRACT

The present disclosure relates to the field of biomedicine, in particular to a drug delivery system for delivering a drug to cells. In an example of the present disclosure, the drug delivery system includes a drug delivery carrier, where a nucleic acid drug and a photothermal nanoparticle are encapsulated in the drug delivery carrier. The drug delivery system opens up an unprecedented means of intracellular drug delivery. Moreover, all materials of the system are desirable in biocompatibility, friendly to the environment without pollution, and harmless during use. Furthermore, compared with other operating methods, a use method of the system is simpler and more convenient to operate. The system shows low production and application costs and is economical-friendly.

### REFERENCE TO SEQUENCE LISTING

A computer readable XML file entitled “GWP20230503728_seqlist”, that was created on Aug. 17, 2023, with a file size of about 3,894 bytes, contains the sequence listing for this application, has been filed with this application, and is hereby incorporated by reference in its entirety.

### TECHNICAL FIELD

The present disclosure relates to the field of biomedicine, in particular to a drug delivery system for delivering a drug to cells, and a preparation method and a use method thereof.

### BACKGROUND

Small-molecule transdermal drug delivery is to deliver small-molecule drugs through a skin barrier into the body to produce pharmaceutical effects. However, many drugs need to enter cells to express related proteins to produce corresponding pharmaceutical effects. These drugs mainly include but not limited to nucleic acid drugs. Currently, methods for delivering such drugs into cells include lipofection, electroporation, gene gun, and viral transfection. Liposomes have been extensively studied as delivery carriers in vivo and in vitro. Nucleic acid drugs encapsulated in synthetic liposomes can also enter human cells through fusion. The electroporation refers to a technique in which small holes or openings are temporarily formed on the cell membrane under the action of an electric field, and nucleic acid drugs enter cells through such small holes or openings. The gene gun is to temporarily form some pores in the cell membrane using high-pressure gas as an accelerating force, and pushes nucleic acid drugs coated on a surface of gold particles into the host cells. The viral transfection is to construct a virus vector, infect a host cell with the virus vector, and integrate a gene into the host cell.

### SUMMARY

In view of this, the technical problem to be solved by the present disclosure is to overcome the defects of low transfection efficiency, relatively complicated operation during preparation, and high application cost in the prior art when delivering nucleic acid drugs to cells.

On one hand, an example of the present disclosure provides a drug delivery system, including a drug delivery carrier, where a nucleic acid drug and a photothermal nanoparticle are encapsulated in the drug delivery carrier.

Optionally, the nucleic acid drug is selected from the group consisting of a DNA drug or an RNA drug.

Optionally, the system further includes an infrared laser emitter, where the infrared laser emitter has an emission wavelength of 760 nm to 1 mm.

Optionally, the infrared laser emitter is a near infrared laser emitter; and the infrared laser emitter has an emission wavelength of 760 nm to 2,526 nm and an illumination density of 3 W/cm² to 4 W/cm².

Optionally, the infrared laser emitter has an emission wavelength of 808 nm and an illumination density of 4 W/cm².

Optionally, the photothermal nanoparticle is selected from the group consisting of a compound of a metal and a non-metal, a metal nanoparticle, and a non-metal nanoparticle.

Optionally, the compound of the metal and the non-metal is Mxene; the metal nanoparticle is selected from the group consisting of a gold nanoparticle and a platinum nanoparticle; and the non-metal nanoparticle is selected from the group consisting of polydopamine (PDA) and graphene oxide (GO).

Optionally, the drug delivery carrier is a microneedle patch.

Optionally, 100 ng to 600 ng of the nucleic acid drug is encapsulated based on a microneedle patch of a 10*10 array.

Optionally, the microneedle patch includes raw materials of polyvinylpyrrolidone (PVP) and hyaluronic acid (HA); the PVP has a molecular weight of 8 kDa to 11 kDa, and the HA has a molecular weight of 3.9 WDa; and the PVP and the HA are at a mass fraction ratio of (10-30):3.

Optionally, the PVP and the HA are at a mass fraction ratio of 20:3.

Optionally, a microneedle patch backing is prepared from PVP with a molecular weight of 32 WDa to 38 WDa.

On the other hand, an example of the present disclosure provides a preparation method of a drug delivery system, where the drug delivery system includes a microneedle patch encapsulating a nucleic acid drug and a photothermal nanoparticle; and
the preparation method includes the following steps:
S1: preparing a needle tip working solution, including:
S11: dissolving raw materials of the microneedle patch to form a mixed solution 1; and
S12: adding a photothermal nanoparticle solution and a nucleic acid drug solution into the mixed solution 1 obtained in step S11, and mixing evenly to obtain the needle tip working solution;
S2: preparing a backing working solution; and
S3: adding the needle tip working solution and the backing working solution into a mold in sequence, and conducting drying, curing, and molding to obtain the microneedle patch.

On the other hand, an example of the present disclosure further provides a use method of a drug delivery system, where the drug delivery system includes a microneedle patch encapsulating a nucleic acid drug and a photothermal nanoparticle; and the use method includes the following steps:
T1: placing the microneedle patch on an administration site of a recipient;
T2: irradiating the microneedle patch with an infrared laser light source, such that the photothermal nanoparticle generates heat until the administration site reaches 50° C. to 80° C.; and
T3: maintaining the administration site at 50° C. to 80° C. for 1 min and then cooling for 1 min, conducting the previous operations for 5 to 10 cycles to facilitate penetration of the nucleic acid drug into the administration site.

The technical solutions of the present disclosure have the following advantages:
1. The present disclosure provides a system for delivering a drug to cells. This photothermal effect-based intracellular drug delivery system creates an unprecedented way of intracellular drug delivery. Moreover, all materials during use are materials with desirable biocompatibility, environmental friendliness, and harmlessness.
2. The present disclosure provides a system for delivering a drug to cells. Compared with other operation methods, a use method of the photothermal effect-based intracellular drug delivery system is simpler and more convenient to operate. The system shows low production and application costs and is economical-friendly.
3. The present disclosure provides a system for delivering a drug to cells. The system has a wide range of applications, not only can be applied to the skin and muscles, but also can be used locally on other organs in theory.

### BRIEF DESCRIPTION OF THE DRAWINGS

To describe the technical solutions in the specific implementations of the present disclosure or the prior art more clearly, the accompanying drawings required for describing the specific implementations or the prior art are briefly described below. Apparently, the accompanying drawings in the following description show merely some implementations of the present disclosure, and a person of ordinary skill in the art may still derive other accompanying drawings from these accompanying drawings without creative efforts.

FIG. 1 shows a structural schematic diagram of a microneedle patch in an example of the present disclosure; where 2 represents a microneedle patch backing, and 1 represents the microneedle patch arranged on the microneedle patch backing;

FIG. 2 shows a bright field photo and an electron micrograph of the microneedle patch in the example of the present disclosure;

FIG. 3 shows a schematic flow chart of a mouse experiment in the example of the present disclosure;

FIG. 4 shows an observation result of a small animal fluorescence imager in the example of the present disclosure;

FIG. 5 shows a result of observing mouse skin with a fluorescence microscope in the example of the present disclosure;

FIG. 6 shows a result of observing the skin under a fluorescence microscope after a skin section is obtained in the example of the present disclosure; where arrows represent where positive cells are located; MN+GPC represents a result of the microneedle patch encapsulating a nucleic acid drug; MN+Mxene represents a result of the microneedle patch encapsulating photothermal nanoparticles; and MN+Mxene+GPC represents a result of the microneedle patch encapsulating the nucleic acid drug and the photothermal nanoparticles;

FIG. 7 shows a schematic flow chart of a use method of the drug delivery system in the example of the present disclosure; and

FIG. 8 shows a graph illustrating experimental results of the nucleic acid drug entering a cell membrane at different temperatures in the drug delivery system in the example of the present disclosure.

### DETAILED DESCRIPTION OF THE EMBODIMENTS

The following examples serve to provide further appreciation of the present disclosure, but are not limited to the preferred examples and do not limit the spirit and scope of the disclosure; any product that is the same as or similar to the disclosure made in light of the disclosure or by combination of the present disclosure with other features of the prior art shall fall within the scope of the disclosure.

If specific experimental procedure or conditions are not indicated in the present disclosure, operations or conditions of conventional experimental procedures known in the art shall be used. If manufacturers of reagents and apparatus used are not indicated, conventional reagent products may be commercially available.

An example of the present disclosure provides a drug delivery system for delivering a drug to cells, including a drug delivery carrier, where a nucleic acid drug and a photothermal nanoparticle are encapsulated in the drug delivery carrier.

The nucleic acid drug can be a DNA drug or an RNA drug.

In the example of the present disclosure, the drug delivery system further includes an infrared laser emitter, where the infrared laser emitter has adjustable power; and the infrared laser emitter has an emission wavelength of 760 nm to 1 mm. In one embodiment, the infrared laser emitter can be a near infrared laser emitter, with an emission wavelength of 760 nm to 2,526 nm and an illumination density of (3-4) W/cm², further preferably an emission wavelength of 808 nm and an illumination density of 4 W/cm².

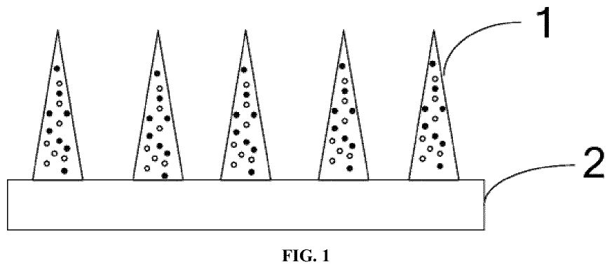

In an example of the present disclosure, the drug delivery carrier may be a microneedle patch. As shown in FIG. 1, the drug delivery carrier includes a microneedle patch backing 2 and the microneedle patch 1 arranged on the microneedle patch backing. Materials that are non-toxic, rapidly dissolving, desirable in biocompatibility, and capable of carrying nucleic acid drugs can be used as a material of the microneedle patch. The material of the microneedle patch includes PVP and HA; the PVP has a molecular weight of (8-11) kDa, and the HA has a molecular weight of 3.9 WDa. The PVP and the HA are at a mass fraction ratio of (10-30):3, preferably 20:3 in this example.

The microneedle patch backing is prepared from PVP with a molecular weight of (32-38) WDa.

An amount of the nucleic acid drug can be controlled according to the addition of drugs with different concentrations; the nucleic acid drug is a DNA drug or an RNA drug. In this example, the nucleic acid drug is loaded with a plasmid expressing a green fluorescent protein (GFP) (plasmid GPC, with a nucleic acid sequence shown in SEQ ID NO: 1).

In an example of the present disclosure, the photothermal nanoparticle is selected from the group consisting of a compound of a metal and a non-metal, a metal nanoparticle, and a non-metal nanoparticle. Optionally, the compound of the metal and the non-metal is Mxene; the metal nanoparticle is selected from the group consisting of a gold nanoparticle and a platinum nanoparticle; and the non-metal nanoparticle is selected from the group consisting of PDA and GO. The photothermal nanoparticle can be the Mxene (Shandong Xiyan New Material Technology Co., Ltd.).

In this example, a microneedle patch of a 10*10 array is prepared. The height of the microneedle patch is 670 μm, the size of the needle tip is 15 μm, the size of the bottom of the microneedle is 300*300 μm. The microneedle patch is a quadrangular pyramid, the distance between the centers of two adjacent microneedles is 500 μm, the size of the backing is 9.8*9.8 mm, and a backing thickness is 1.5 mm. The specific size and shape of the microneedle can be changed according to experimental needs. Each microneedle patch (the microneedle patch of the 10*10 array) encapsulates a nucleic acid drug (in the experiment, a plasmid that encodes GFP, namely a plasmid GPC) in an amount of 500 ng, and encapsulates nanoparticles (in this example, Mxene) in an amount of 536 ng. The specific amounts of nucleic acid drug and nanoparticles can be changed according to experimental needs. The microneedle backing material is PVP with a molecular weight of (32-38) WDa.

500 ng of the plasmid GPC is encapsulated in each 10*10 array of microneedle patch.

The example of the present disclosure further provides a preparation method of the drug delivery system. Take a preparation method of a microneedle patch encapsulating a nucleic acid drug and a photothermal nanoparticle as an example, the preparation method was described as follows.

### Preparation of Materials

A configuration method of a 1 g/ml PVP solution included:
5 g of powdered PVP was put into a test tube, added with deionized water to 5 ml, shaken for 20 min to fully dissolve the PVP to obtain the 1 g/ml PVP solution.

A configuration method of a 150 mg/ml HA solution included:
750 mg of HA was put into a test tube, added with deionized water to 5 ml, shaken for 20 min to fully dissolve the HA to obtain the 150 mg/ml HA solution.

### Preparation of Microneedle Patch

Step I: a microneedle material was selected.

Microneedle patch materials included PVP with a molecular weight of (8-11) kDa at a concentration of 1 g/ml and HA with a molecular weight of 3.9 WDa at a concentration of 150 mg/ml. A microneedle backing working solution was PVP with a molecular weight of (32-38) WDa.

Step II: a microneedle working solution was prepared, including a needle tip (i.e.) working solution and a backing working solution.

The microneedle backing working solution was a PVP solution with a concentration of 0.5 g/ml.

The needle tip working solution was described by taking the preparation of 400 μl of the needle tip working solution as an example. A PVP solution, an HA solution, and a photothermal nanoparticle solution were at a volume ratio of (2-4):(2-4):1. Specifically,
1) 133.92 μl of the 1 g/ml PVP solution was mixed with 133.92 μl of the 150 mg/ml HA solution, shaken fully to mix uniformly, put into a vacuum pump for 30 min, taken out, and bubbles in the solution were removed to obtain a mixed solution 1.
2) Mxene and a plasmid GPC expressing GPC were added to the mixture 1 as required. The needle tip working solution was to ensure that 500 ng of the plasmid GPC was encapsulated in each microneedle patch. Since a needle tip part of a PDMS concave mold had a volume of 2.01 μl, in order to ensure that 500 ng of GPC was encapsulated in each microneedle patch, a concentration of GPC in the needle tip working solution was:

$$\frac{500}{2.01} \approx 248.8 ng / µl$$

Therefore, 66.96 μl of a 1.6 mg/ml Mxene solution and 65.2 μl of a 1335.2 ng/μl GPC solution were added to the mixed solution 1, shaken fully, and vacuumed to remove air bubbles in the working solution, to obtain a uniform needle tip working solution.

Step III: after the two working solutions were prepared, the PDMS concave mold was removed to start making the microneedle.

The needle tip working solution was pipetted into the PDMS concave mold with a pipette gun, where it was necessary to ensure that the needle tip working solution covered a pinhole array of the mold. Vacuum extraction was conducted in a vacuum pump with a pressure of −103 kPa for 3 h. During the extraction, the inside of the vacuum pump needed to be humidified to avoid water loss in the working solution during the extraction. After the extraction was completed, the mold was put on a stereoscope to observe. If there were a few air bubbles at the edge, they could be picked out manually with a pipette. If there were more residual air bubbles, it was necessary to continue vacuumizing until the working solution filled the needle tip, and the excess working solution was recovered.

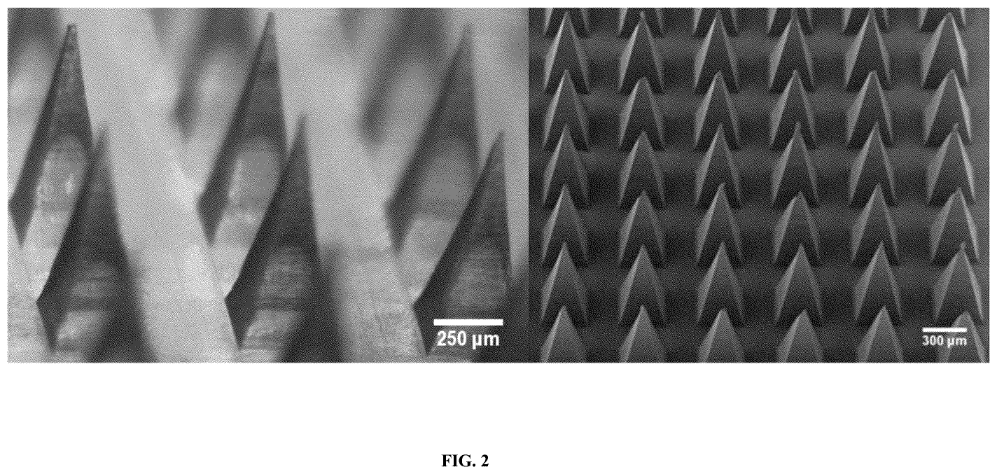

Step IV: the backing working solution was added into the PDMS concave mold, such that the backing solution covered and protruded the mold surface to form an arc, so as to avoid backing defects caused by water loss during the curing. The mold was dried and cured for 24 h at 20° C. and 15% air humidity for molding. The microneedle patch encapsulating the nucleic acid drug and photothermal nanoparticles was peeled off with tweezers or adhesive tape, as shown in FIG. 2.

### Use Method of the Microneedle Patch

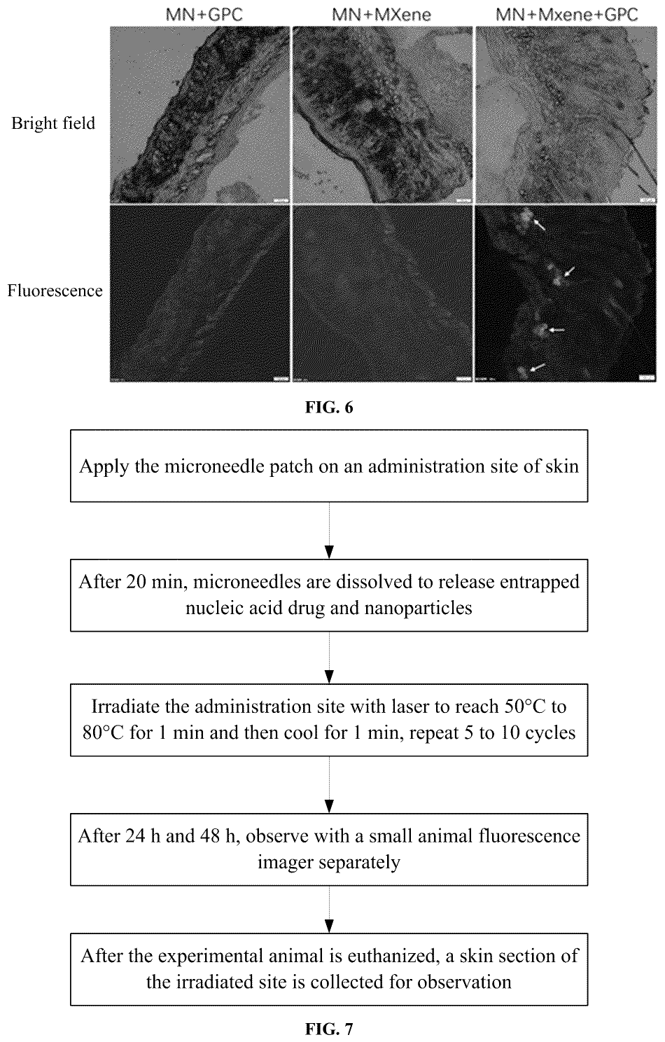

Regarding the drug delivery system, the example of the present disclosure further provided a use method of the drug delivery system, as shown in FIG. 7. The use method included:
T1: placing the microneedle patch on an administration site of a recipient;
T2: irradiating the microneedle patch with an infrared laser light source, such that the photothermal nanoparticle generates heat until the administration site reaches 50° C. to 80° C.; and
T3: maintaining the administration site at 50° C. to 80° C. for 1 min and then cooling for 1 min, conducting the previous operations for 5 to 10 cycles to facilitate penetration of the nucleic acid drug into the administration site.

### EXAMPLE & COMPARATIVE EXAMPLE

Infrared laser light source irradiation was conducted to heat the photothermal nanoparticles in the microneedle patch. The photothermal nanoparticles conducted heat to the cell membrane, causing local arrangement of the cell membrane to be disordered, thereby allowing the drug to enter the cells.

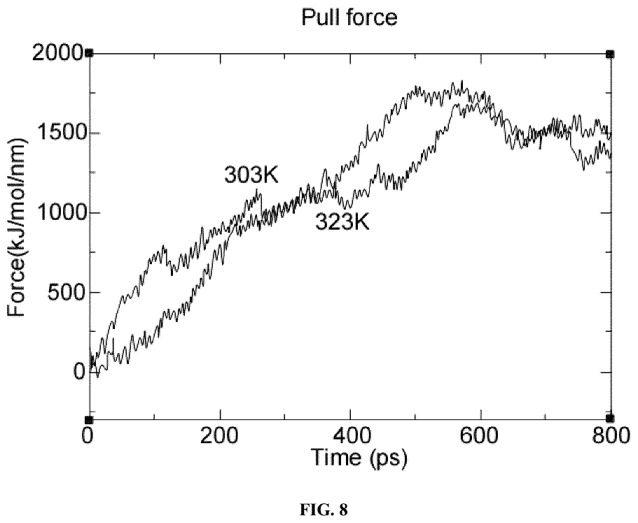

As shown in the molecular dynamics simulation experiment results of FIG. 8, nucleic acid drugs (such as DNA) showed average resistance in penetrating the cell membrane at 323 K (50° C.) that was smaller than that at 303 K (30° C.). That is, DNA penetrated the cell membrane more easily at 50° C.

According to the research of the present disclosure, it was found that when the administration site was maintained at 50° C. to 80° C., the nucleic acid drug could quickly enter the cell membrane without causing damage to the cells.

### Animal Experiment

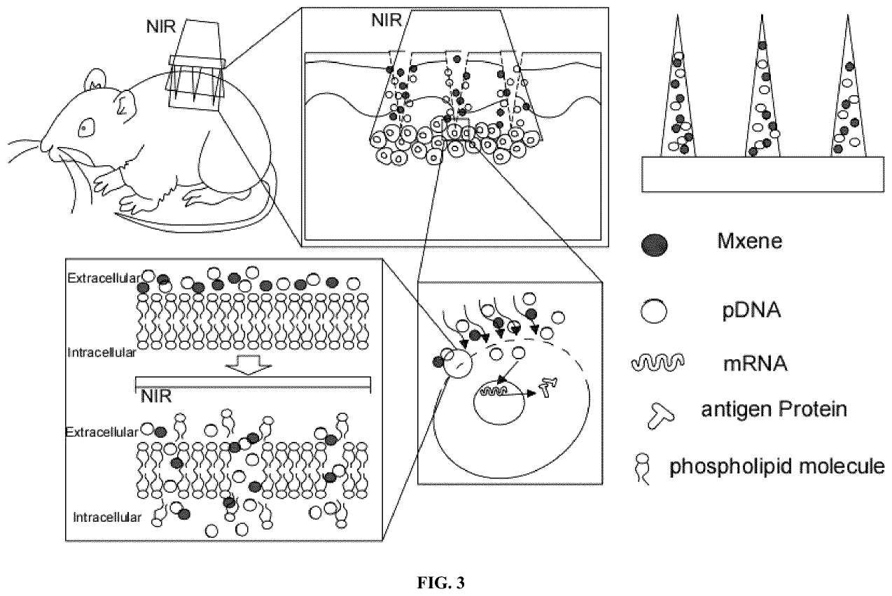

1. Combining the mouse experiment shown in FIG. 3, experimental mice C57BL/6N were anesthetized, and their back was partially depilated. The microneedle patch encapsulating the nucleic acid drug and the photothermal nanoparticles was pierced into the depilated part of the back of the mice. After 20 min, the microneedle backing was irradiated with a near-infrared laser in vivo at an emission wavelength of 808 nm and an illumination density of 4 W/cm² for 1 min. After an interval of 1 min, irradiation was conducted again, and the above operations were conducted for 5 cycles. The nucleic acid drug in the microneedle patch encapsulating the nucleic acid drug and photothermal nanoparticles was replaced with deionized water as a control.

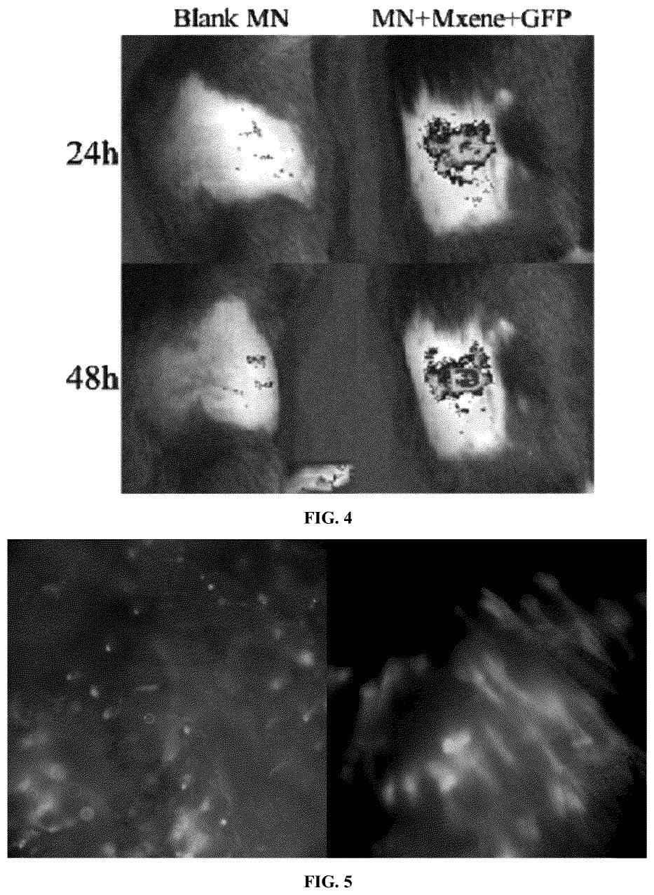

2. At the end of this experiment, data collection was required, first of all, observation was conducted with a small animal fluorescence imager. After the end of experiment, the mice were observed at 24 h and 48 h once separately. The experimental mice were anesthetized with isoflurane before use. The software was started, and relevant settings were adjusted. A wavelength of the excitation light was set to 480 nm, a wavelength of the emitted light was set to 520 nm, and an exposure time was adjusted as needed; in this experiment, the exposure time was set to 200 s; the Binning was set to 2, the Fstop was set to 2, and then data were collected after photographing. The results were shown in FIG. 4. In the figure, blank MN represented a control result, while MN+Mxene+GFP represented a result of the microneedle patch encapsulating nucleic acid drug and photothermal nanoparticles. After the data collection was completed, the software was used to adjust the experimental image, such as a luminescent type of the fluorescent part and the setting of a fluorescent threshold in the image, to make the experimental comparison more beautiful. Through the results, it was seen that the MN+Mxene+GFP group showed stronger fluorescence than the blank MN group, suggesting that the microneedle-treated part had GFP expression. This indicated that the GPC plasmid had been delivered into the cells to express GFP. The results of observing the skin with a fluorescence microscope were shown in FIG. 5. Finally, the mouse skin was sectioned and observed at the cellular level. As shown in FIG. 6, it was seen that the expression of GFP was consistent with the spacing of the microneedle.

It is apparent that the above embodiments are merely listed for clear description, and are not intended to limit the implementations. The person of ordinary skill in the art may make modifications or variations in other forms based on the above description. There are no need and no way to exhaust all the implementations. Obvious changes or variations made thereto shall still fall within the protection scope of the present disclosure.

### Sequence Listing Information

DTD Version: V1_3
File Name: GWP20230503728_seqlist.xml
Software Name: WIPO Sequence
Software Version: 2.3.0
Production Date: 2023 Aug. 17

### General Information

Current application/Applicant file reference: GWP20230503728
Applicant name: BEIHANG UNIVERSITY
Applicant name/Language: en
Invention title: DRUG DELIVERY SYSTEM, AND PREPARATION METHOD AND USE METHOD THEREOF (en)
Sequence Total Quantity: 1

### Sequences:

Sequence Number (ID): 1
Length: 2217
Molecule Type: DNA
Features Location/Qualifiers:
   source, 1 . . . 2217
      mol_type, other DNA
      note, Plasmid GPC
      organism, synthetic construct

### Residues

| | |
|---|---|
| ggatccatgg gacaaatagt gacattcttc caggaagtgc ctcatgtaat agaagaggtg | 60 |
| atgaacattg ttctcattgc actgtctgta ctagcagtgc tgaaaggtct gtacaatttt | 120 |
| gcaacgtgtg gccttgttgg tttggtcact ttcctcctgt tgtgtggtag gtcttgcaca | 180 |
| accagtcttt ataaaggggt ttatgagctt cagactctgg aactaaacat ggagacactc | 240 |
| aatatgacca tgcctctctc ctgcacaaag aacaacagtc atcattatat aatggtgggc | 300 |
| aatgagacag gactagaact gaccttgacc aacacgagca ttattaatca caaattttgc | 360 |
| aatctgtctg atgcccacaa aaagaacctc tatgaccacg ctcttatgag cataatctca | 420 |
| actttccact tgtccatccc caacttcaat cagtatgagg caatgagctg cgattttaat | 480 |
| gggggaaaga ttagtgtgca gtacaacctg agtcacagct atgctgggga tgcagccaac | 540 |
| cattgtggta ctgttgcaaa tggtgtgtta cagactttta tgaggatggc ttggggtggg | 600 |
| agctacattg ctcttgactc aggccgtggc aactgggact gtattatgac tagttatcaa | 660 |
| tatctgataa tccaaaatac aacctgggaa gatcactgcc aattctcgag accatctccc | 720 |
| atcggttatc tcgggctcct ctcacaaagg actagagata tttatattag tagaagattg | 780 |
| ctaggcacat tcacatggac actgtcagat tctgaaggta aagacacacc agggggatat | 840 |
| tgtctgacca ggtggatgct aattgaggct gaactaaaat gcttcgggaa cacagctgtg | 900 |
| gcaaaatgta atgagaagca tgatgaggaa ttttgtgaca tgctgaggct gtttgacttc | 960 |
| aacaaacaag ccattcaaag gttgaaagct gaagcacaaa tgagcattca gttgatcaac | 1020 |
| aaagcagtaa atgctttgat aaatgaccaa cttataatga agaaccatct acgggacatc | 1080 |
| atgggaattc catactgtaa ttacagcaag tattggtacc tcaaccacac aactactggg | 1140 |
| agaacatcac tgcccaaatg ttggcttgta tcaaatggtt catacttgaa cgagacccac | 1200 |
| ttttctgatg atattgaaca acaagctgac aatatgatca ctgagatgtt acagaaggag | 1260 |
| tatatggaga ggcaggggaa gacaccattg ggtctagttg acctctttgt gttcagtaca | 1320 |
| agtttctatc ttattagcat cttccttcac ctagtcaaaa taccaactca taggcatatt | 1380 |
| gtaggcaagt cgtgtcccaa acctcacaga ttgaatcata tgggcatttg ttcctgtgga | 1440 |
| ctctacaaac agcctggtgt gcctgtgaaa tggaagagag gcggtggcgg tatggtgagc | 1500 |
| aagggcgagg agctgttcac cggggtggtg cccatcctgg tcgagctgga cggcgacgta | 1560 |
| aacggccaca agttcagcgt gtccggcgag ggcgagggcg atgccaccta cggcaagctg | 1620 |
| accctgaagt tcatctgcac caccggcaag ctgcccgtgc cctggcccac cctcgtgacc | 1680 |
| accctgacct acggcgtgca gtgcttcagc cgctaccccg accacatgaa gcagcacgac | 1740 |
| ttcttcaagt ccgccatgcc cgaaggctac gtccaggagc gcaccatctt cttcaaggac | 1800 |
| gacggcaact acaagacccg cgccgaggtg aagttcgagg gcgacaccct ggtgaaccgc | 1860 |
| atcgagctga agggcatcga cttcaaggag gacggcaaca tcctggggca caagctggag | 1920 |
| tacaactaca acagccacaa cgtctatatc atggccgaca agcagaagaa cggcatcaag | 1980 |
| gtgaacttca agatccgcca caacatcgag gacggcagcg tgcagctcgc cgaccactac | 2040 |
| cagcagaaca cccccatcgg cgacggcccc gtgctgctgc ccgacaacca ctacctgagc | 2100 |
| acccagtccg ccctgagcaa agaccccaac gagaagcgcg atcacatggt cctgctggag | 2160 |
| ttcgtgaccg ccgccgggat cactctcggc atggacgagc tgtacaagta agatatc | 2217 |

What is claimed is:

1. A drug delivery system, comprising a drug delivery carrier, wherein a nucleic acid drug and a photothermal nanoparticle are encapsulated in the drug delivery carrier.

2. The system according to claim 1, wherein the nucleic acid drug is selected from the group consisting of a DNA drug or an RNA drug.

3. The system according to claim 1, further comprising an infrared laser emitter, wherein the infrared laser emitter has an emission wavelength of 760 nm to 1 mm.

4. The system according to claim 2, further comprising an infrared laser emitter, wherein the infrared laser emitter has an emission wavelength of 760 nm to 1 mm.

5. The system according to claim 3, wherein the infrared laser emitter is a near infrared laser emitter; and the infrared laser emitter has an emission wavelength of 760 nm to 2,526 nm and an illumination density of 3 W/cm² to 4 W/cm².

6. The system according to claim 4, wherein the infrared laser emitter is a near infrared laser emitter; and the infrared laser emitter has an emission wavelength of 760 nm to 2,526 nm and an illumination density of 3 W/cm² to 4 W/cm².

7. The system according to claim 5, wherein the infrared laser emitter has an emission wavelength of 808 nm and an illumination density of 4 W/cm².

8. The system according to claim 6, wherein the infrared laser emitter has an emission wavelength of 808 nm and an illumination density of 4 W/cm².

9. The system according to claim 1, wherein the photothermal nanoparticle is selected from the group consisting of a compound of a metal and a non-metal, a metal nanoparticle, and a non-metal nanoparticle.

10. The system according to claim 2, wherein the photothermal nanoparticle is selected from the group consisting of a compound of a metal and a non-metal, a metal nanoparticle, and a non-metal nanoparticle.

11. The system according to claim 9, wherein
the compound of the metal and the non-metal is Mxene; the metal nanoparticle is selected from the group consisting of a gold nanoparticle and a platinum nanoparticle; and the non-metal nanoparticle is selected from the group consisting of polydopamine (PDA) and graphene oxide (GO).

12. The system according to claim 10, wherein
the compound of the metal and the non-metal is Mxene; the metal nanoparticle is selected from the group consisting of a gold nanoparticle and a platinum nanoparticle; and the non-metal nanoparticle is selected from the group consisting of polydopamine (PDA) and graphene oxide (GO).

13. The system according to claim 1, wherein the drug delivery carrier is a microneedle patch.

14. The system according to claim 2, wherein the drug delivery carrier is a microneedle patch.

15. The system according to claim 9, wherein
100 ng to 600 ng of the nucleic acid drug is encapsulated in the drug delivery carrier based on a microneedle patch of a 10*10 array.

16. The system according to claim 13, wherein the microneedle patch comprises raw materials of polyvinylpyrrolidone (PVP) and hyaluronic acid (HA); the PVP has a molecular weight of 8 kDa to 11 kDa, and the HA has a molecular weight of 3.9 WDa; and the PVP and the HA are at a mass fraction ratio of (10-30):3.

17. The system according to claim 16, wherein the PVP and the HA are at a mass fraction ratio of 20:3.

18. The system according to claim 13, wherein a microneedle patch backing is prepared from PVP with a molecular weight of 32 WDa to 38 WDa.

19. A preparation method of a drug delivery system, wherein the drug delivery system comprises a microneedle patch encapsulating a nucleic acid drug and a photothermal nanoparticle; and
the preparation method comprises the following steps:
S1: preparing a needle tip working solution, specifically comprising:
S11: dissolving raw materials of the microneedle patch to form a mixed solution 1; and
S12: adding a photothermal nanoparticle solution and a nucleic acid drug solution into the mixed solution 1 obtained in step S11, and mixing evenly to obtain the needle tip working solution;
S2: preparing a backing working solution; and
S3: adding the needle tip working solution and the backing working solution into a mold in sequence, and conducting drying, curing, and molding to obtain the microneedle patch.

20. A use method of a drug delivery system, wherein the drug delivery system comprises a microneedle patch encapsulating a nucleic acid drug and a photothermal nanoparticle; and
the use method comprises the following steps:
T1: placing the microneedle patch on an administration site of a recipient;
T2: irradiating the microneedle patch with an infrared laser light source, such that the photothermal nanoparticle generates heat until the administration site reaches 50° C. to 80° C.; and
T3: maintaining the administration site at 50° C. to 80° C. for 1 min and then cooling for 1 min, conducting the previous operations for 5 to 10 cycles to facilitate penetration of the nucleic acid drug into the administration site.
